(19) United States
(12) Patent Application Publication
(10) Pub. No.: US 2013/0253448 A1
(43) Pub. Date: Sep. 26, 2013

(54) Hypodermic Needle With Multiple Dispersement Openings

(71) Applicants: Steven Baron, Lawrence, NY (US); Richard Arote, Lynbrook, NY (US)

(72) Inventors: Steven Baron, Lawrence, NY (US); Richard Arote, Lynbrook, NY (US)

(21) Appl. No.: 13/787,962

(22) Filed: Mar. 7, 2013

Related U.S. Application Data
(60) Provisional application No. 61/608,830, filed on Mar. 9, 2012.

(51) Int. Cl. A61M 5/32

(52) U.S. Cl. CPC A61M 5/3291
USPC 604/272

(57) ABSTRACT

A needle used for injecting viscous materials into a patient is provided. The needle would attach to a syringe. The needle end, although pointed, is blocked or stopped so that no fluid may exit. Along the barrel of the needle is a plurality of openings through which the viscous material or other medicament would be discharged into the patient. These openings may be circular, elliptical, square, rectangular or other geometric shapes. Certain sizes of the apertures and distance from one aperture to another are disclosed.

### PRIORITY APPLICATIONS

This Utility Application claims priority from the Provisional Patent Application Ser. No. 61/608,830 filed on Mar. 9, 2012.

### BACKGROUND OF THE INVENTION

The present invention relates to the injection of drugs and other fluids, that are highly viscous, into a patient, where it is desirable to evenly distribute the fluid over a relatively small area in proximity to the point of injection.

Specifically, dermal fillers are used in a relatively small target area to smooth the wrinkles of the face and/or to augment other facial areas such as lips to create a more attractive appearance, by adding volume and fullness to the skin to correct moderate to severe facial wrinkles and folds such as lines from the nose to the corners of the mouth as well as lip enhancement. Typical dosages of these dermal fillers are 0.6 mL in wrinkles and folds, and 1.5 mL per lip.

### BRIEF SUMMARY OF THE INVENTION

The present invention is an improved hypodermic needle constructed and arranged such that the needle has multiple openings to evenly distribute a viscous drug or other material over a relatively small area in proximity to the point of injection.

Procedures during and after plastic surgery recommend use of soft tissue dermal filler fluids to add volume. These fillers are highly viscous and are injected at a specific location in or under a layer of the skin. These fluids may for example include Restylane (a trademark of Medics Aesthetics, Inc., Scottsdale Ariz.), Juvaderm (a trademark owned by Allergan Industrie SAS and Allergan, Inc. Irvine, Calif.), Radiesse (a trademark of Merz Aesthetics, Inc. San Mateo, Calif.) and other fillers, which are injected with a hypodermic needle. It is often difficult to discharge these fluids, because of their high viscosity, through the openings in the hypodermic needle and distribute the fluid in an even manner over the area of the skin where the dermal filler is to be injected and deposited.

There are several difficulties in discharging the dermal filler during the procedure. These include at least the following: a constant high force or pressure is required to be applied against the plunger of the hypodermic needle by the operator to force the fluid out of the needle; and the difficulty of distributing the viscous fluid through a single outlet at the terminal end of the needle.

### BRIEF DESCRIPTION OF THE DRAWINGS

FIG. 1A is a plan view of a hypodermic needle.

FIG. 1B is a sectional view of a hypodermic needle taken from lines C-C from FIG. 1A.

FIG. 1C is a perspective view of a hypodermic needle of the present invention shown as it is inserted into the facial area of a patient during the process of injection a fluid.

FIG. 2A is a plan view of a needle of the present invention.

FIG. 2B is a sectional view of the needle shown in FIG. 2A taken along lines D-D.

FIG. 2C is a sectional view of the needle shown in FIG. 2A along lines E-E.

FIG. 3A is a plan view of an alternate needle of the present invention.

FIG. 3B is a sectional view of the needle shown in FIG. 3A taken along lines F-F.

FIG. 3C is a sectional view of the needle shown in FIG. 3A taken along lines H-H.

FIG. 3D is a sectional view of the needle shown in FIG. 3A taken along lines G-G.

FIG. 4 is a plan view of a portion of facial skin showing an area having a wrinkle or wrinkle line or other area to receive treatment.

FIG. 5 is section view along lines B-B of FIG. 6.

FIG. 6 is a plan view of a wrinkle line section of skin similar to FIG. 4 with a needle shown injected into the skin area.

FIG. 7A is a cross section view along lines I-I of FIG. 7B of an alternative needle shown in FIG. 7B having several openings and the distal end closed with different sized and shaped openings.

FIG. 7B is a plan view of the needle shown in FIG. 7A.

FIG. 7C is a cross section view along lines J-J of the needle in FIG. 7B showing a cross section of the hole and barrel of the needle.

FIG. 8A is a plan view of another alternative needle having square openings along its length with the distal end closed.

FIG. 8B is an enlarged view of detail A from FIG. 8A.

FIG. 9A is a plan view of yet another alternative needle having rectangular openings along its length with the distal end closed.

FIG. 9B is an enlarged view of detail B from FIG. 9A.

### DETAILED DESCRIPTION OF THE PREFERRED EMBODIMENT

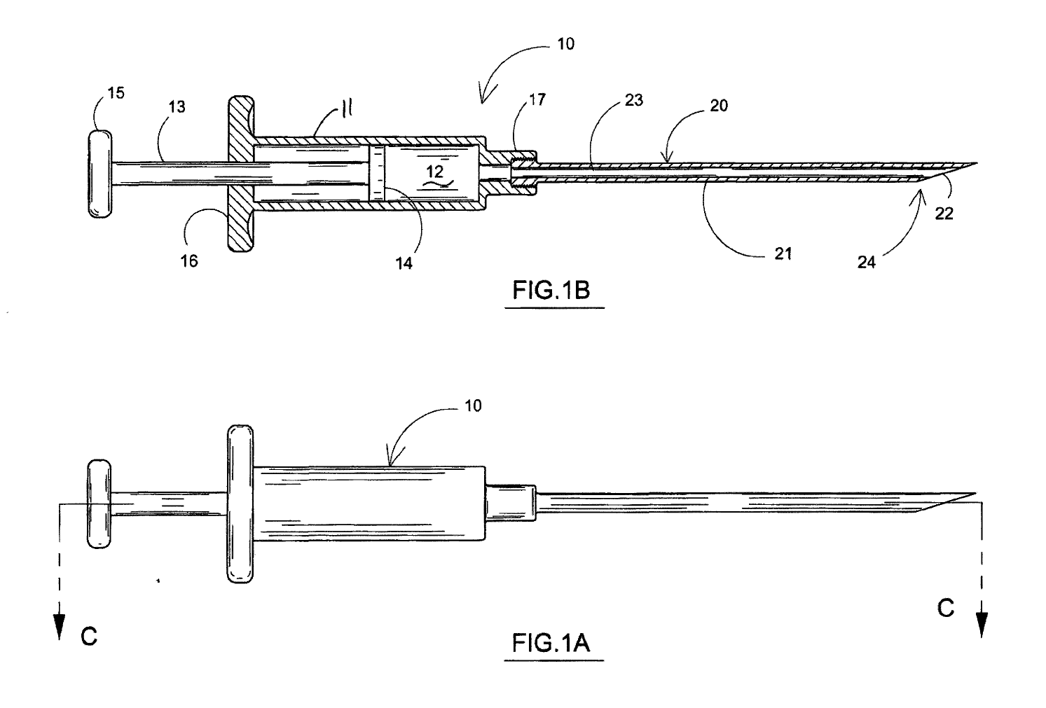

The present invention is an improved needle which would be a part of and typically used with a hypodermic needle. As seen in FIG. 1A, hypodermic needle 10 has a barrel 11 which typically would be of glass or other clear material in order to see the contents within barrel 11. The contents would typically be a fluid 12. A plunger 13 slides within barrel 11 which has at the distal end a plunger head 14 which as known in the art keeps the fluid 12 at the distal side of head 14 within barrel 11. A thumb rest 15 is at the proximal end of plunger 13 and a finger flange 16 is located at the proximal end of barrel 11. A needle 20 with a beveled tip 24 is attached to the distal end of barrel 13 at needle hub 17. Needle 20 is a hollow tube having an inner wall 23 within needle 20. Typically needle 20 is made of steel and fluidly communicates with barrel 11 such that when the plunger 13 is urged in the direction of needle 20 any fluid 12 within barrel 11 will be forced out the distal end of barrel 11, through hub 17 and through hollow needle 20 and disbursed out the distal end 24 of needle 20 at an orifice or opening 22.

As seen in FIG. 1A, a typical hypodermic needle 10 as known in the art, has a needle 20. The needle 20 is hollow having a needle barrel 21, with a single opening 22 at the distal end 24 of needle 20.

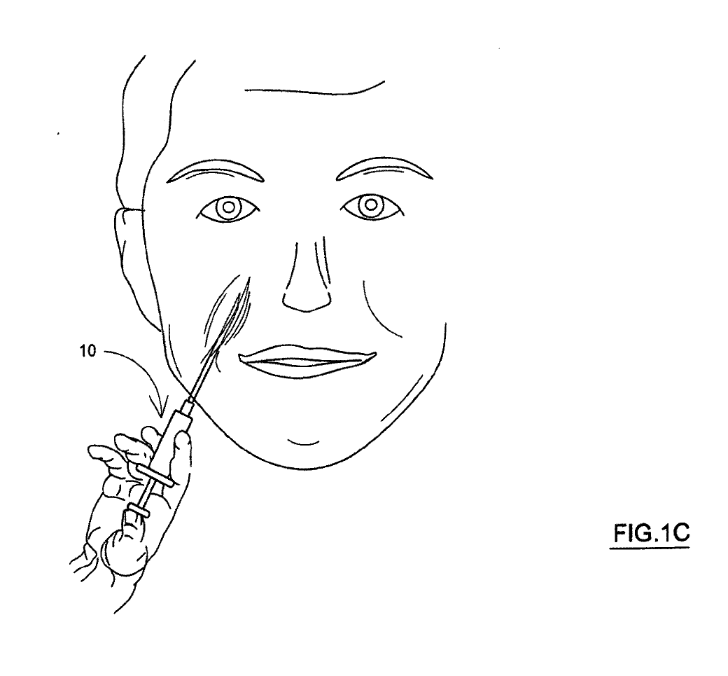

The needle 20 of the present invention as seen in FIG. 2A, provides for multiple openings or holes 25 in the needle barrel 21 of needle 20. Thus the fluid 12 within hypodermic needle 10 is discharged through multiple openings 25 to evenly distribute the fluid 12 in the desired deposit area. As seen in FIG. 2A, the openings 25 are arranged in a single row and are spaced apart along the length of needle 20 and there is the distal opening 22 at the distal end 24. FIG. 2B shows a sectional view of needle 20 taken along lines D-D from FIG. 2A and further shows the openings 25 in the needle barrel 21 portion of needle 20. FIG. 2C is a cross sectional view of an opening 25 taken along lines E-E from FIG. 2A.

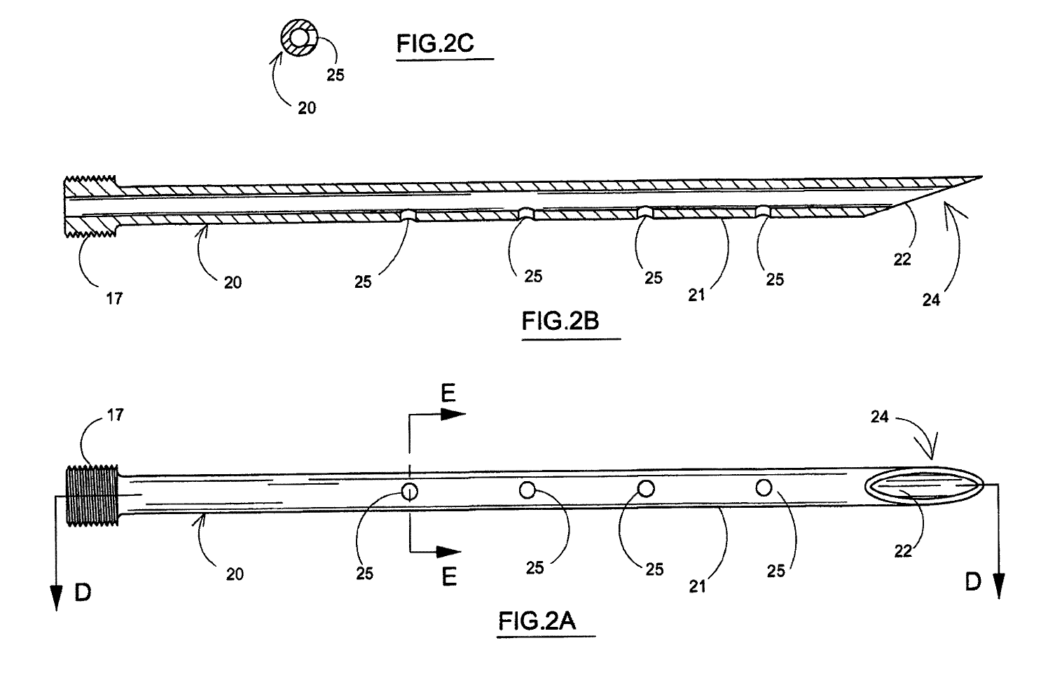

In an alternate embodiment shown in FIG. 3A, needle 20 has three rows of openings 25 where each row is spaced 120 degrees apart from the other adjacent row, and spaced equally around the needle barrel 21 of needle 20. As in the prior embodiment, there is a terminal end 24 and opening 22. The needles 20 in FIGS. 2A and 3A may or may not have an opening 22 at terminal end 24, since there are other openings 25 in the needle 20. Further, the openings 25 may have different diameters with the openings 25 closest to hub 17 having smaller diameters than those closer to the distal end 24 in view of the internal pressures to cause the fluid 12 to flow evenly out of the several openings at the same time.

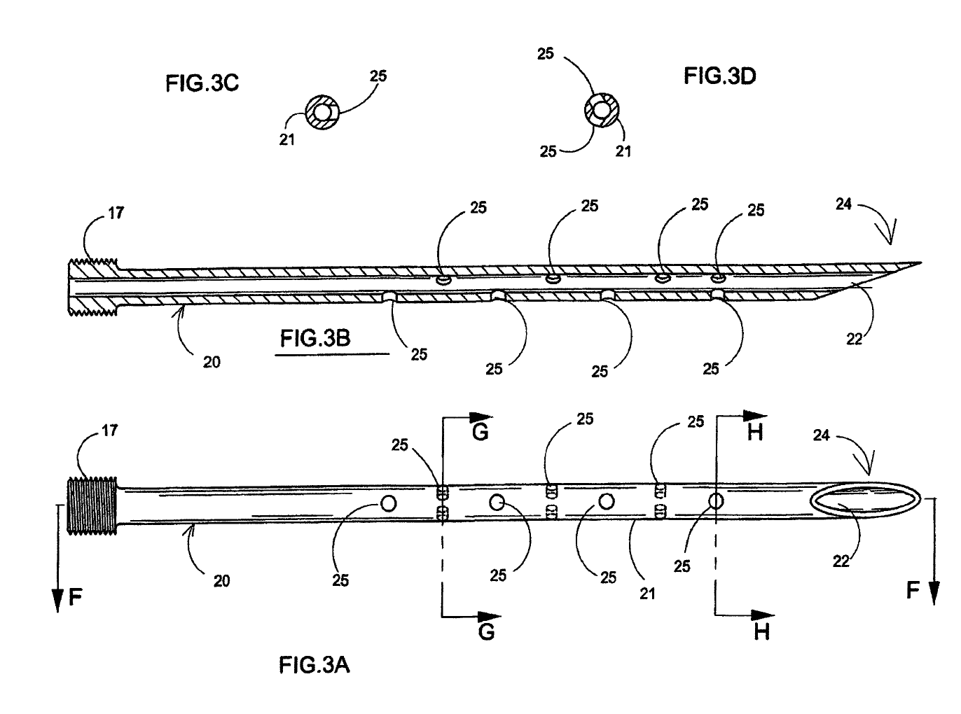

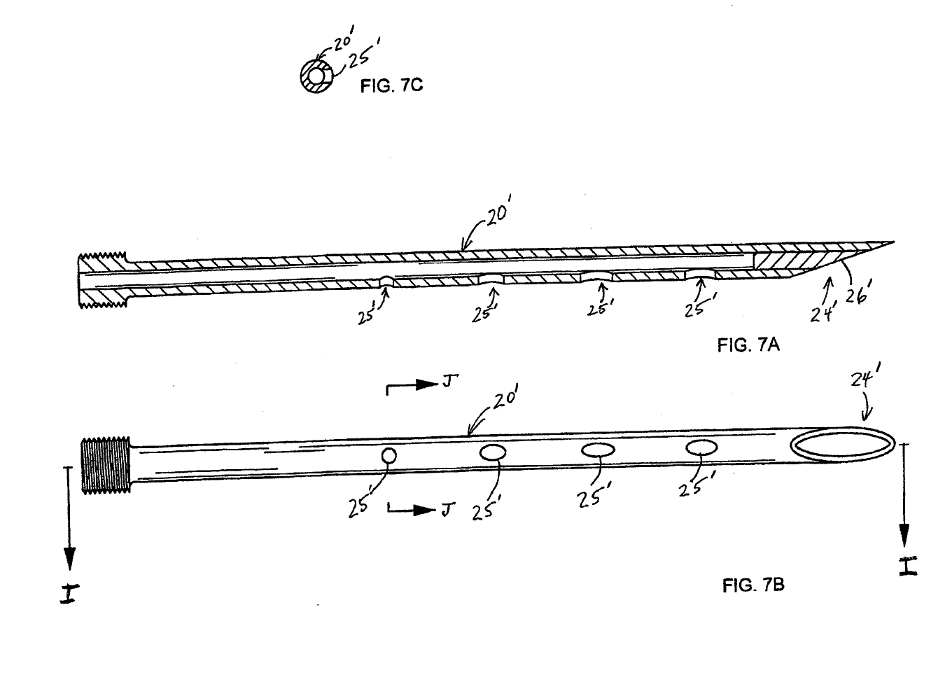

In use, it would be desired to inject a filler fluid 12 into a location or area of a wrinkle line 40 in the facial skin of a patient. As seen in FIGS. 4 and 1C, plan views of an area of a wrinkle line 40 extends from a first end 42 to a second end 43, with a portion of skin 41 that includes the wrinkle 40. Though the FIGS. 4 and 6 show wrinkle line 40 to be relatively straight, the wrinkle line 40 could also be curved or have a generally jagged length. Cosmetically it is desirable to fill the area below the wrinkle line 40 with a filler to at least have it appear that the wrinkle line 40 is no longer visible. The filler is a viscous fluid 12 and would be injected under the skin generally by use of hypodermic needle 10. Typically, as known in the art, it is desirable to evenly distribute the viscous fluid 12 along wrinkle line 40.

In the prior art, a hypodermic needle 10 would be filled with a viscous fluid, and the needle 20 would be inserted into an injection opening site 30 at a first end 42 of the wrinkle line 40. Wrinkle line 40 may extend from a point 42 to point 43, a distance marked as A. The needle 20 would be inserted into point 42 at one end of the wrinkle line 40 under the skin 41 and the needle would extend along the wrinkle line 40 to the end of the wrinkle line 43. Then, the person applying the filler would push the plunger 13 to force the viscous fluid 12 out of the single opening 22 of needle 20 and simultaneously begin withdrawing the needle 20 from its injection point 30, so that the fluid 12 is evenly disbursed and distributed along the wrinkle line 40 below the skin 41 in an even manner. In other words, the desired result would be to have the needle 20 withdrawn from the opening 30 at the injection site, such that the filler fluid 12 has been evenly distributed along the wrinkle line 40. However, this is extremely difficult in practice, since the operation of the plunger 13 by pushing the thumb rest 15 is very tiring and difficult since the viscous fluid 12 is so difficult to push out of the opening 22 of needle 20.

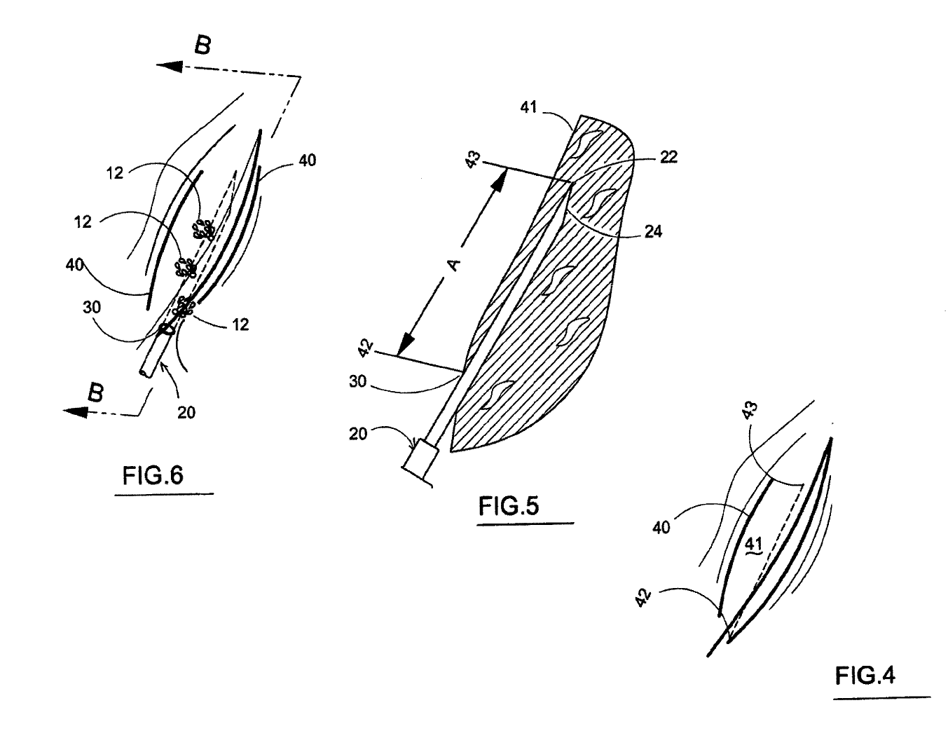

The advantage of the present invention, by having multiple openings 25 in needle 20, the needle 20 is inserted at the injection site 30 extending the entire length of the wrinkle line 40 or a desired portion of the wrinkle line 40. The needle 20 with multiple openings 25 can effectively be used to discharge the entire desired amount of fluid 12 easily through the multiple openings 25 without having to move the needle 20 during the discharge of the fluid 12. Because the openings 25 are evenly spaced, the fluid 12 is disbursed in the desired locations evenly through the multiple openings. FIG. 5 shows in cross section the needle 20 extending below the skin from the injection site 30 a length A from the first opening 42 to the second opening 43. As seen in FIG. 6 with the needle in the position shown in FIG. 5, the fluid 12 can be disbursed from the needle without withdrawing the needle and the fluid 12 will be disbursed evenly about the wrinkle line 40 as shown in FIG. 6.

In alternate embodiments, it is possible the wrinkle line 40 will be sufficiently longer than needle 20 and therefore, the above steps may be repeated. In this manner, the wrinkle line 40 may be twice or three times the length of the needle 20. In this situation, there would be additional injection locations along the wrinkle line 40.

In addition, it is known that the injection of fluids through a needle into the body often causes pain. It is believed the pain is caused likely not only by the insertion of the needle tip 24 into and puncturing the skin, but also as a result of the infusion of the fluid into the body e.g. the muscle where the tip of the needle 22 is located and where the fluid 12 leaves the needle 20 and enters the body. It is now believed that the improved needle tip 22 of the present invention provides a better method of infusing fluids through a needle and into the body by dispersing the fluid not through a single opening of the needle but through several openings of the needle as described herein. The dispersion through a plurality of openings is believed to likely reduce the pain that occurs to the patient when the fluids are injected through a single opening needle.

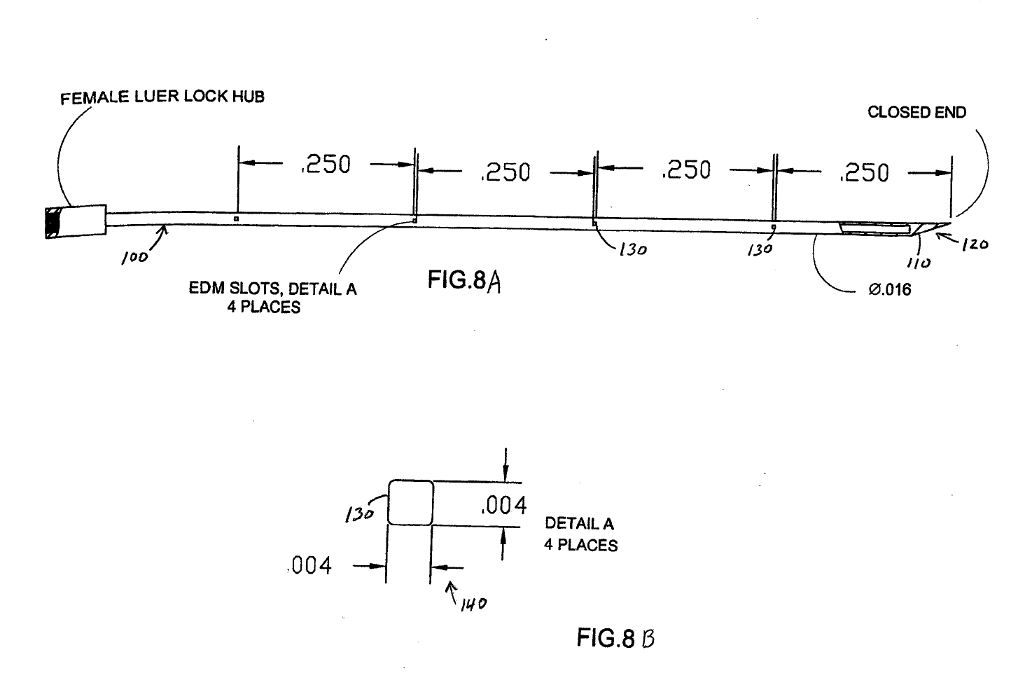

As shown in FIG. 7A is a needle 20′ with alternate embodiments, including a plug 26′ or other sealing means to close the terminal end 24′ of needle 20′. In addition openings 25′ are shown in different sizes and shapes to facilitate tuning of the needle 20′, so that fluid 12′ will disperse from the multiple openings 25′ in an even and equal manner. Alternatively, the openings 25′ may be such that fluid 12′ will disperse in an unequal manner depending on the requirements of the plastic surgery.

FIG. 7B is a plan view of the needle 20′ showing the plurality of openings 25′ with various geometrical configurations. These configurations include, but are not limited to, a circle or an ellipse with an increasing eccentricity. The greater the eccentricity the longer the semi-major axis is, thus producing an ellipse which is longer and thinner.

FIG. 7C is a cross section of the needle 20′ in FIG. 7B taken at line J-J showing a cross section of the hole 25′ and barrel of the needle 20′.

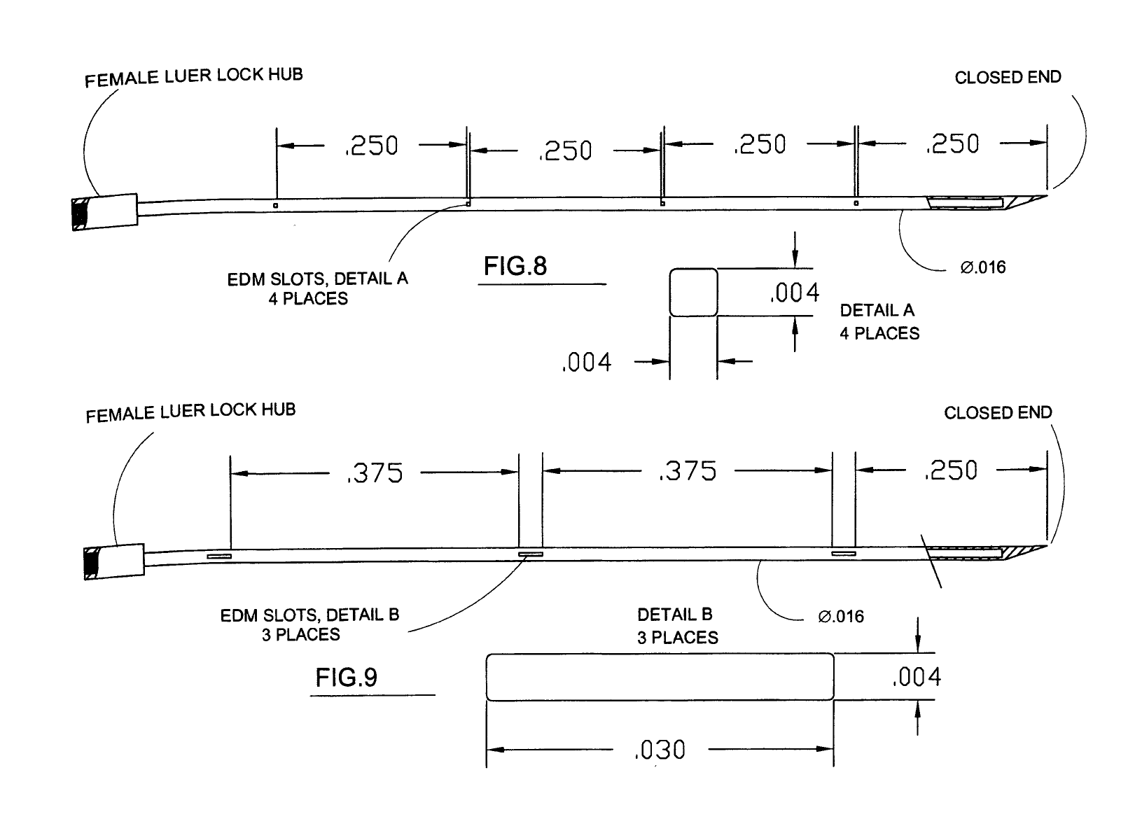

FIG. 8A is a plan view of still another embodiment of the needle 100. Needle 100 includes a plug 110 or other sealing means to close the terminal end 120 of needle 100. Along the barrel of the needle 100 are a plurality of square apertures 130.

In this particular embodiment the square apertures 130 are separated from each other by 0.25 of an inch. The square apertures of this embodiment further measure 0.004 of an inch square, each side of the square is 0.004 inch. A spotlight blowup 140 of one of the square apertures 130 is shown. The dimensions of the square apertures 130 may vary, and although not shown, one square aperture 130 may have a different side length making the square aperture 130 either larger or smaller. In the embodiment shown in FIG. 8A, there are four identical square apertures 130. It can easily be seen that each of the square apertures 130 may be of a different size, or two having one size and the other two having a second size. Also, although four square apertures 130 are shown, the number and size of each of them may vary due to specific application.

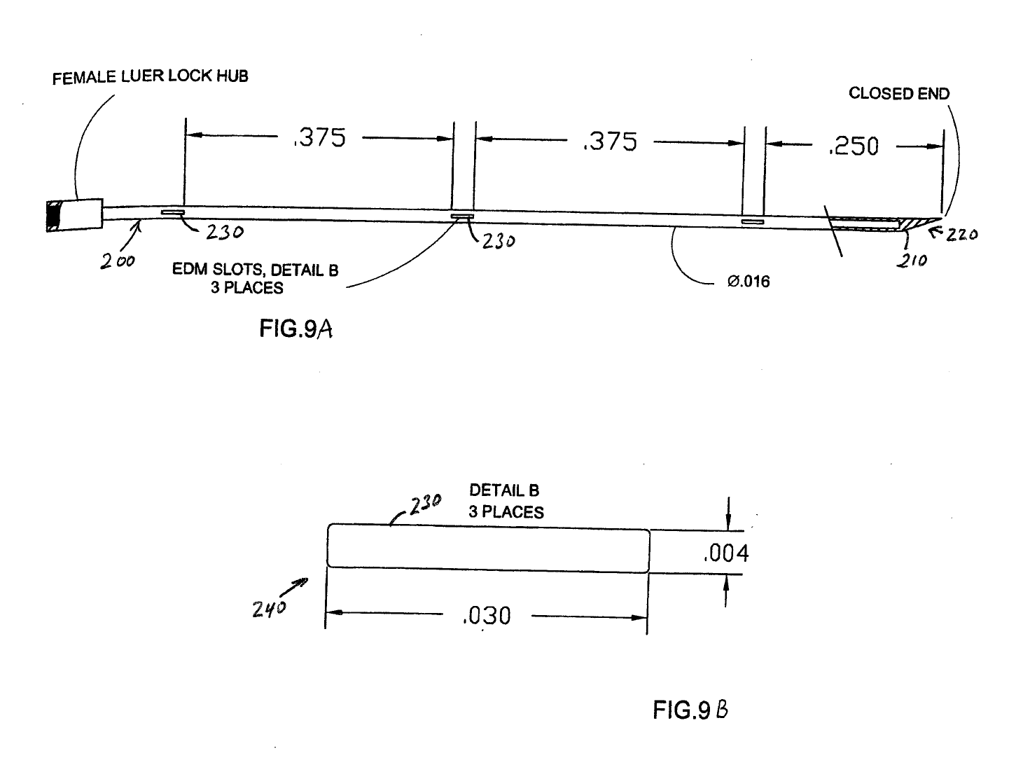

FIG. 9A is a plan view of still another embodiment of the needle 200. Needle 200 includes a plug 210 or other sealing means to close the terminal end 220 of needle 200. Along the barrel of the needle 200 are a plurality of rectangular apertures 230.

In this particular embodiment the rectangular apertures 230 are separated from each other by 0.375 of an inch. The rectangular apertures of this embodiment further measure 0.004 of an inch on one side of the rectangle, and 0.030 of an inch on the other side of the rectangle. A spotlight blowup 240 of one of the rectangular apertures 230 is shown. The dimensions of the rectangular apertures 230 may vary, and although not shown, one rectangular aperture 230 may have a different side length on one side or the second side making the rectangular aperture 230 either larger or smaller. In the embodiment shown in FIG. 9A, there are three identical rectangular apertures 230. It can easily be seen that each of the rectangular apertures 230 may be of a different size, or two having one size and the other one having a second size or visa versa. Also, although three rectangular apertures 230 are shown, the number and size of each of them may vary due to specific application.

The distance from one aperture to another in all embodiments may be in the range of 0.001 inch to 1.0 inch. The diameter of the circular apertures may be in the range of 0.001 to 0.5 inch. The semi-major axis of the elliptical aperture may be in the range of 0.001 to 0.5 inch. The semi-minor axis of the elliptical aperture may be in the range of 0.001 inch to 0.5 inch. The square aperture may have a side distance of 0.0001 to 0.5 inch. The rectangular aperture long side distance may be of 0.0001 inch to 0.5 inch. The rectangular aperture short side distance may be in the range of 0.0001 inch to 0.5 inch. In the case where other geometrical apertures are employed, the range of size would be equivalent to the range of the square aperture, which size would permit an equivalent amount of viscous material through.

While the invention has been described in its preferred form or embodiment with some degree of particularity, it is understood that this description has been given only by way of example and that numerous changes in the details of construction, fabrication, and use, including the combination and arrangement of parts, may be made without departing from the spirit and scope of the invention.

1. A hypodermic needle having a barrel for holding a fluid to be dispersed through a needle and a plunger for applying force to the fluid to disperse the fluid comprising:
a hollow needle attachable to a hypodermic needle, said hypodermic needle having a barrel for storing a fluid and a plunger for pushing said fluid from said barrel out said needle,
said needle having a first end and a second end and said first end attached to said barrel,
said needle having a center bore terminating in an opening at said second end, and
said needle having one or more openings along the side of said needle.

2. A hypodermic needle as in claim 1 wherein said openings along the side of said needle are aligned longitudinally along the length of said needle.

3. A hypodermic needle as in claim 2 wherein said openings along the side of said needle are aligned longitudinally along two or more rows along the length of said needle.

4. A hypodermic needle as in claim 2 wherein the second end of said needle is closed.

5. A hypodermic needle as in claim 2 wherein said openings along the side of said needle have different sized and shaped openings.

6. A hypodermic needle as in claim 5 wherein said openings along the side of said needle have the shape of a rectangle.

7. A hypodermic needle as in claim 5 wherein said openings along the side of said needle have the shape of an ellipse.

8. A hypodermic needle as claimed in claim 5 wherein said openings along the side of said needle have the shape of a square.

9. A hypodermic needle as claimed in claim 5 wherein said openings along the side of said needle are of different geometrical configurations, said geometrical configurations to be chosen from the group of circles, ellipses, squares or rectangles.
